(19) United States
(12) Patent Application Publication
(10) Pub. No.: US 2013/0317412 A1
(43) Pub. Date: Nov. 28, 2013

(54) Flow Control For Treating A Medical Condition

(71) Applicants: Bruno Dacquay, Irvine, CA (US); Cesario P. Dos Santos, Aliso Viejo, CA (US); Casey J. Lind, Orange, CA (US); Robert J. Sanchez, JR., Oceanside, CA (US); Mathew J.A. Rickard, Yorba Linda, CA (US)

(72) Inventors: Bruno Dacquay, Irvine, CA (US); Cesario P. Dos Santos, Aliso Viejo, CA (US); Casey J. Lind, Orange, CA (US); Robert J. Sanchez, JR., Oceanside, CA (US); Mathew J.A. Rickard, Yorba Linda, CA (US)

(21) Appl. No.: 13/478,145

(22) Filed: May 23, 2012

(51) Int. Cl. A61F 9/00

(52) U.S. Cl. 604/9

(57) ABSTRACT

An apparatus for treatment of a medical condition of a patient to provide drainage from a region of a patient to a drainage location includes a memory having a plurality of different selectable treatment algorithms stored therein for treating a medical condition, and may include a processor associated with the memory and configured to execute one of the different treatment algorithms. It also may include a flow system controllable by the processor to regulate drainage of fluid from a body portion having a medical condition, the flow system being controllable according to a selected algorithm of said different selectable treatment algorithms. In one aspect, the apparatus further comprises an implantable medical device for treating an ocular condition, and the processor is carried on the implantable medical device.

### BACKGROUND

The present disclosure relates generally to pressure/flow control systems and methods for use in treating a medical condition. In some instances, embodiments of the present disclosure are configured to be part of an IOP control system for the treatment of ophthalmic conditions.

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

The eye's ciliary body continuously produces aqueous humor, the clear fluid that fills the anterior segment of the eye (the space between the cornea and lens). The aqueous humor flows out of the anterior chamber (the space between the cornea and iris) through the trabecular meshwork and the uveoscleral pathways, both of which contribute to the aqueous drainage system. The delicate balance between the production and drainage of aqueous humor determines the eye's IOP.

FIG. 1 is a diagram of the front portion of an eye that helps to explain the processes of glaucoma. In FIG. 1, representations of the lens 110, cornea 120, iris 130, ciliary body 140, trabecular meshwork 150, Schlemm's canal 160, and the anterior chamber 170 are pictured. Anatomically, the anterior segment of the eye includes the structures that cause elevated IOP which may lead to glaucoma. Aqueous fluid is produced by the ciliary body 140 that lies beneath the iris 130 and adjacent to the lens 110 in the anterior chamber 170 of the anterior segment of the eye. This aqueous humor washes over the lens 110 and iris 130 and flows to the drainage system located in the angle of the anterior chamber 170. The angle of the anterior chamber 170, which extends circumferentially around the eye, contains structures that allow the aqueous humor to drain. The trabecular meshwork 150 is commonly implicated in glaucoma. The trabecular meshwork 150 extends circumferentially around the anterior chamber. The trabecular meshwork 150 seems to act as a filter, limiting the outflow of aqueous humor and providing a back pressure that directly relates to IOP. Schlemm's canal 160 is located beyond the trabecular meshwork 150. Schlemm's canal 160 is fluidically coupled to collector channels (not shown) allowing aqueous humor to flow out of the anterior chamber 170. The two arrows in the anterior segment of FIG. 1 show the flow of aqueous humor from the ciliary bodies 140, over the lens 110, over the iris 130, through the trabecular meshwork 150, and into Schlemm's canal 160 and its collector channels.

One method of treating glaucoma includes implanting a drainage device in a patient's eye. The drainage device allows fluid to flow from the interior chamber of the eye to a drainage site, relieving pressure in the eye and thus lowering TOP. These devices are generally passive devices and do not provide a smart, interactive control of the amount of flow through the drainage tube.

The system and methods disclosed herein overcome one or more of the deficiencies of the prior art.

### SUMMARY

In one exemplary aspect, the present disclosure is directed to an apparatus for treatment of a medical condition of a patient to provide drainage from a region of a patient to a drainage location. The apparatus may include a memory having a plurality of different selectable treatment algorithms stored therein for treating a medical condition, and may include a processor associated with the memory and configured to execute one of the different treatment algorithms. It also may include a flow system controllable by the processor to regulate drainage of fluid from a body portion having a medical condition, the flow system being controllable according to a selected algorithm of said different selectable treatment algorithms.

In one aspect, the apparatus further comprises an implantable medical device for treating an ocular condition, and the processor is carried on the implantable medical device. In one aspect, each of the plurality of treatment algorithms comprises a plurality of periods of time and target settings corresponding to the periods of time. In one aspect, the pressure/flow system comprises one of a valve and a pump, and the processor controls a setting on said valve or pump.

In another exemplary aspect, the present disclosure is directed to a control system for treatment of an ocular condition of a patient to provide drainage from an anterior chamber of the eye to a drainage location. The control system may include a memory having a plurality of different selectable treatment algorithms stored therein for treating an ocular condition. It also may include a processor associated with the memory and configured to execute one of the different treatment algorithms. A sensor system may be configured to detect a pressure representative of an anterior chamber and configured to detect a pressure representative of atmospheric pressure. The processor may be configured to generate control signals based on a selected one of the different treatment algorithms and on the detected pressures.

In one aspect, the apparatus includes a flow system comprising one of a valve and a pump controllable by the processor to regulate drainage of fluid from an eye, the flow system being controllable according to said selected one of said different selectable treatment algorithms.

In another exemplary aspect, the present disclosure is directed to a method comprising: storing a plurality of selectable treatment algorithms in a memory;

receiving an input selecting one of the selectable treatment algorithms of the plurality of selectable treatment algorithms in a memory; and controlling a flow system to regulate drainage of fluid from a body portion having a medical condition based on the selected one of the selectable treatment algorithms.

In one aspect, the method comprises incrementally adjusting the treatment algorithm. In one aspect, the treatment algorithm includes a target IOP level and or the rate of change from the current setpoint. In another aspect, the treatment algorithm comprises a target open amount of an adjustable valve and or the rate of change from the current setpoint.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of the front portion of an eye.

FIG. 2 is a block diagram of an exemplary IOP control system according to the principles of the present disclosure.

FIG. 3 is a schematic diagram of an exemplary implant including the IOP control system of FIG. 2 disposed on an eye according to the principles of the present disclosure.

FIG. 4 is a flow chart of an exemplary method of choosing a treatment algorithm according to one embodiment consistent with the principles of the present disclosure.

FIG. 5 is a graph showing an exemplary selectable treatment algorithm for treating an ocular condition according to one embodiment consistent with the principles of the present disclosure.

FIG. 6 is a flow chart of an exemplary method of using a treatment algorithm to treat an ocular condition according to one embodiment consistent with the principles of the present disclosure.

FIG. 7 is another graph showing an exemplary selectable treatment algorithm for treating an ocular condition according to one embodiment consistent with the principles of the present disclosure.

FIG. 8 is yet another graph showing an exemplary selectable treatment algorithm for treating an ocular condition according to one embodiment consistent with the principles of the present disclosure.

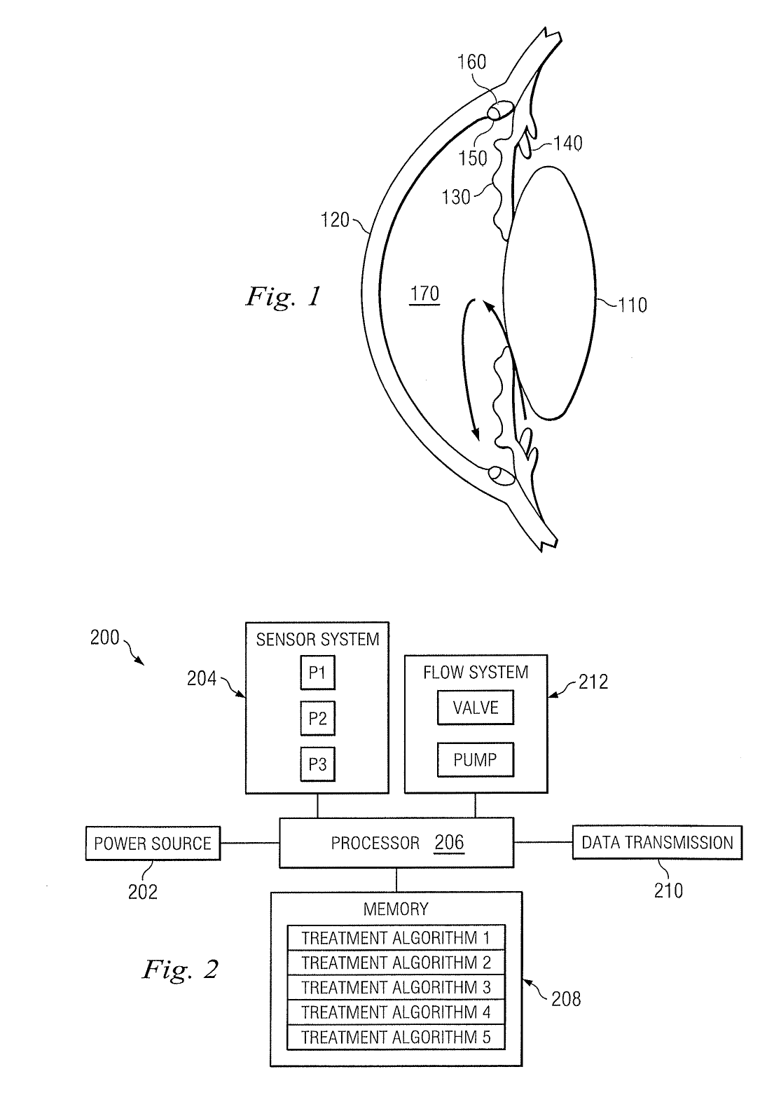

### DETAILED DESCRIPTION OF THE PREFERRED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure is directed to a flow control system for treating a medical condition, such as glaucoma. In one aspect, the system controls IOP by regulating fluid drainage through an implant such as a glaucoma drainage device (GDD). The system may control the drainage based on a stored treatment algorithm that provides a desired treatment to a patient to control IOP. In one aspect, the system includes a plurality of stored treatment algorithms and a health care provider may select a desired treatment algorithm from the plurality of stored treatment algorithms based on any number of factors, including surgeon preference, the nature of the treatment, patient characteristics, such as age, size, or overall health, and other factors. Once selected, the treatment algorithm controls the implant to provide a particular treatment profile based on the selected treatment algorithm. Permitting a health care provider to select a particular treatment algorithm may result in better treatment, a more consistent recovery, and ultimately a better patient outcome.

FIG. 2 is a block diagram of an exemplary IOP control system 200 usable as a part of a device implantable in an eye of a patient for the treatment of glaucoma or other conditions. In FIG. 2, the IOP control system 200 includes a power source 202, an IOP sensor system 204, a processor 206, memory 208, a data transmission module 210, and a flow system 212.

The power source 202 is typically a rechargeable battery, such as a lithium ion or lithium polymer battery, although other types of batteries may be employed. In addition, any other type of power cell is appropriate for power source 202. Power source 202 provides power to the system 200, and more particularly to processor 206. In one embodiment, the power source can be recharged via inductive coupling such as an RFID link or other type of electromagnetic coupling.

The processor 206 is typically an integrated circuit with power, input, and output pins capable of performing logic functions. In various embodiments, processor 206 is a targeted device controller. In such a case, the processor 206 performs specific control functions targeted to a specific device or component, such as the data transmission module 210, the power source 202, the sensing system 204, the flow system 212, or the memory 208. In other embodiments, the processor 206 is a microprocessor. In such a case, processor 206 is programmable so that it can function to control more than one component of the device. In other cases, processor 206 is not a programmable microprocessor, but instead is a special purpose controller configured to control different components that perform different functions.

The memory 208 is typically a semiconductor memory such as RAM, FRAM, or flash memory. The memory 208 interfaces with the processor 206. As such, the processor 206 can write to and read from the memory 208. For example, processor 206 can be configured to read data from the IOP sensor system 204 and write that data to memory 208. In the embodiments shown, a series of treatment algorithms or treatment profiles are stored in the memory 208 for access and execution by the processor. These treatment algorithms may be selected by a health care provider for execution by the processor 208 to treat a medical condition. While only five treatment algorithms are shown for convenience, any number of algorithms may be stored in the control system for selection by a user. In one aspect, the plurality of algorithms are stored on a device separate from the implant for selection, and then the selected algorithm is transferred to implant to control the implant. It should be noted, that the care provider may also be able to design a specific treatment algorithm and load that to the device using a device separate from the implant. The processor 206 is also capable of performing other basic memory functions, such as erasing or overwriting the memory 208, detecting when memory 208 is full, and other common functions associated with managing memory.

The data transmission module 210 may employ any of a number of different types of data transmission. For example, the data transmission module 210 may be an active device such as a radio. Data transmission module 210 may also be a passive device such as the antenna on an RFID tag. In this case, an RFID tag includes memory 208 and data transmission module 210 in the form of an antenna. An RFID reader can then be placed near the system 200 to write data to or read data from memory 208. Therefore, the treatment algorithms may be transmitted to the memory via the data transmission module, along with any selection of or adjustment to the stored treatment algorithms. Other types of data that can be stored in memory 208 and transmitted by data transmission module 210 include, but are not limited to, IOP measurement data, power source data (e.g. low battery, battery defect), speaker data (warning tones, voices), IOP sensor data (IOP readings, problem conditions), time stamp data and the like.

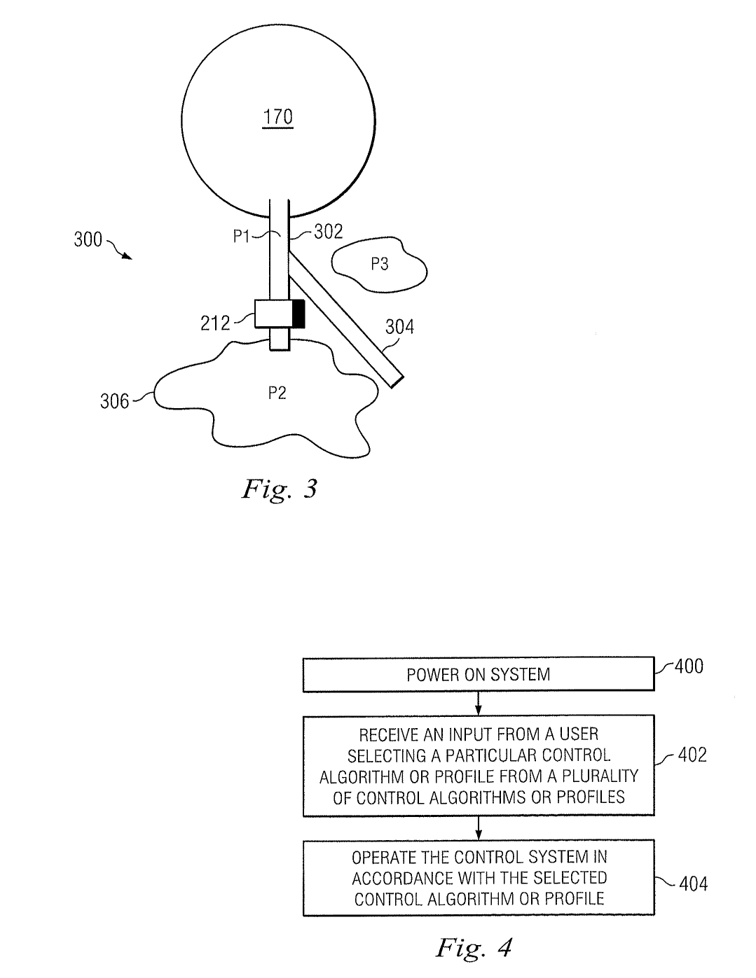

The pressure/flow system 212 may include components or elements that control pressure by regulating the amount of drainage flow. In the example shown, the flow system 212 includes a valve and a pump. The flow system may include any number of valves and any number of pumps, or may not include a pump or may not include a valve. In a preferred embodiment, the flow system 212 is an active system that is responsive to signals from the processor 206 to increase flow, decrease flow, or to maintain a steady flow as a function of pressure. In one embodiment, it does this by maintaining a valve setting at a consistent setting, or increasing or decreasing the amount that the valve is open. The IOP sensor system 204 is described below with reference to FIG. 3.

FIG. 3 is a diagram of the exemplary IOP control system 200 as a part of an implant 300 implanted within an eye of a patient. In this example the implant includes a drainage tube 302 and a divider 304, associated with components of the control system 200. For example, the flow system 212 and the IOP sensor system 204 are identified in FIG. 3. The exemplary IOP sensor system 204 includes three pressure sensors, P1, P2, and P3 (also shown in FIG. 2). Pressure sensor P1 is located in or is in fluidic communication with the anterior chamber (labeled 170), pressure sensor P2 is located at a drainage site (e.g., 306 in FIG. 3) that may be in the subconjunctival space, and pressure sensor P3 is located remotely from P1 and P2 in manner to measure atmospheric pressure. In some embodiments, pressure sensor P1 is located in a lumen or tube that is in fluid communication with the anterior chamber.

The drainage tube 302 drains aqueous from the anterior chamber 170 of the eye. The flow system 212 regulates the flow of aqueous through the tube 302. In the embodiment shown, the pressure sensor P1 measures the pressure in the tube 302 upstream from the flow system 212 and downstream from the anterior chamber 170. In this manner, pressure sensor P1 measures the pressure in the anterior chamber 170. The expected measurement discrepancy between the true anterior chamber pressure and that measured by P1 when located in a tube downstream of the anterior chamber (even when located between the sclera and the conjunctiva) is very minimal. For example, Poiseuille's law for pipe flow predicts a pressure drop of 0.01 mmHg across a 5-millimeter long tube with a 0.300 millimeter inner diameter for a flow rate of 3 microliters per minute of water.

Pressure sensor P2 is located at the drainage site 306. As such, pressure sensor P2 may be located in a pocket, such as a bleb, that generally contains aqueous or in communication with such a pocket, via a tube for example, and is in a wet location 306. The drainage site 306 may be, for example, in a subconjunctival space, a suprachoroidal space, a subscleral space, a supraciliary space, Schlemm's canal, a collector channel, an episcleral vein, and an uveo-scleral pathway, among other locations in the eye.

In some embodiments, the divider 304 acts as a barrier that separates the pressure sensor P3 from the pressure sensor P2. In some embodiments, the system includes other barriers that separate the sensors P1, P2, and P3. These barriers may be elements of the system itself. In FIG. 3, the pressure sensor P3 is physically separated from pressure sensor P2 by the divider 304. Divider 304 is a physical structure that separates the drainage area 306 from the isolated location of P3. In one example, the barrier separating anterior chamber pressure sensor P1 and the drainage site pressure sensor P2 is the flow system 212. In some examples, the IOP control system 200 is formed as a glaucoma drainage device having a plate. In this example, the atmospheric sensor P3 may reside on a top of the plate with a barrier preventing it from being crushed while still allowing pressure communication, such as through the conjunctiva. In other examples atmospheric sensor P3 may be connected to tubing routing to a region exposed to pressures that may be representative of atmospheric pressure. The drainage site sensor P2 may then reside on the bottom in direct contact with the drainage site.

Generally, IOP is a gauge pressure reading—the difference between the absolute pressure in the eye (as measured by P1) and atmospheric pressure (as measured by P3). Atmospheric pressure, typically about 760 mm Hg, often varies in magnitude by 10 mmHg or more depending on weather conditions or indoor climate control systems. In addition, the effective atmospheric pressure can vary significantly—in excess of 200 mmHg—if a patient goes swimming, hiking, riding in an airplane, etc. Such a variation in atmospheric pressure is significant since IOP is typically in the range of about 15 mm Hg. Thus, for accurate monitoring of IOP, it is desirable to have pressure readings for the anterior chamber (as measured by P1) and atmospheric pressure in the vicinity of the eye (as measured by sensor P3).

Therefore, in one embodiment of the present invention, pressure readings are taken by pressure sensors P1 and P3 simultaneously or nearly simultaneously over time so that the actual TOP can be calculated (as P1−P3 or P1−f(P3), where f(P3) indicates a function of P3). The pressure readings of P1 and P3 can be stored in memory 208 by processor 206. They can later be read from memory so that actual IOP over time can be interpreted by a physician.

Pressure sensors P1, P2, and P3 can be any type of pressure sensors suitable for implantation in the eye. They each may be the same type of pressure sensor, or they may be different types of pressure sensors. For example, pressure sensors P1 and P2 may be the same type of pressure sensor (implanted in the eye), and pressure sensor P3 may be a different type of pressure sensor (in the vicinity of the eye).

In another embodiment of the present invention, pressure readings taken by pressure sensors P1, P2, and P3 can be used to control a device that drains aqueous from the anterior chamber 170.

The drainage tube 302 may be arranged to shunt fluid from the anterior chamber 170 to the drainage location 306, which may be placed at any of numerous locations within the eye. For example, some tubes are arranged to shunt aqueous from the anterior chamber 170 to the subconjunctival space thus forming a bleb under the conjunctiva or alternatively, to the subscleral space thus forming a bleb under the sclera. Other tube designs shunt aqueous from the anterior chamber to the suprachoroidal space, the supraciliary space, the juxta-uveal space, or to the choroid, forming blebs in those respective locations. In other applications, the drainage tube shunts aqueous from the anterior chamber to Schlemm's canal, a collector channel in Schlemm's canal, or any of a number of different blood vessels like an episcleral vein. In some examples, the drainage tube even shunts aqueous from the anterior chamber to outside the conjunctiva. Each of these different anatomical locations to which aqueous is shunted is an example of a drainage location 306. Other examples of a drainage location 306 include, but are not limited to: a subconjunctival space, a suprachoroidal space, a subscleral space, a supraciliary space, Schlemm's canal, a collector channel, an episcleral vein, and an uveo-scleral pathway.

One complication involved with surgery that shunts the anterior chamber 170 to a drainage location 306 is hypotony—a dangerous drop in IOP that can result in severe consequences. Accordingly, it can be desirable to control the rate of aqueous outflow from the anterior chamber 170 to the drainage location 306 so as to prevent hypotony.

In one exemplary aspect, the present disclosure is directed to a system that employs a particular treatment plan, as a stored treatment algorithm, to provide therapeutic treatment to the patient, such as to the eye of the patient. In this exemplary aspect, the treatment algorithms dictate the changes to the implant 300, including changes to the flow system 212, that may occur to regulate the IOP and maintain a desired level. In one aspect, the flow system 212 is controlled to open, close, or throttle flow to optimize efficacy. For example according to one stored treatment algorithm or profile, the flow system 212 may include a valve that is closed during an initial period of time to allow the implantation area to heal. The valve may then be opened or throttled at a later time to control IOP. Using the pressure sensor feedback, the valve state may be manipulated such that IOP is kept constant or allowed to ramp up or down. In one aspect, various control algorithms may be implemented at different times of the day to accommodate synergy with a drug therapy or a patient's diurnal patterns.

FIG. 4 shows a method for operating the control system 200 to treat a medical condition. In one aspect, the method is carried out by the implant 300 to treat an ophthalmic condition. In the example in FIG. 4, the method begins when the control system 200, as a part of the implant 300 is powered on, as indicated at step 400. Here, the system 200 has a plurality of pre-stored treatment algorithms selectable by a user. These may be stored in the memory, as shown in FIG. 2 on-board or off-board the implant. At a step 402, the system 200 receives an input from a user selecting a particular treatment algorithm from the plurality of treatment algorithms or profiles. In one example, the system 200 may receive the input directly through a user interface or may receive the input through the data transmission module 210. The user interface may be an element of the implant 300, and may be a part of, or may be connectable to the implant 300. It may include buttons, a display, or a mouse or other input device. When the system 200 receives the input through the data transmission module 210, a separate user interface may be used to receive the user's input, which may then be transmitted to the implant 300. At a step 404, the control system operates the implant in accordance with the selected treatment algorithm or profile. Note that one possible option of control algorithm or profile may be a fully customizable one in which the care giver fully defines all aspects of the selection.

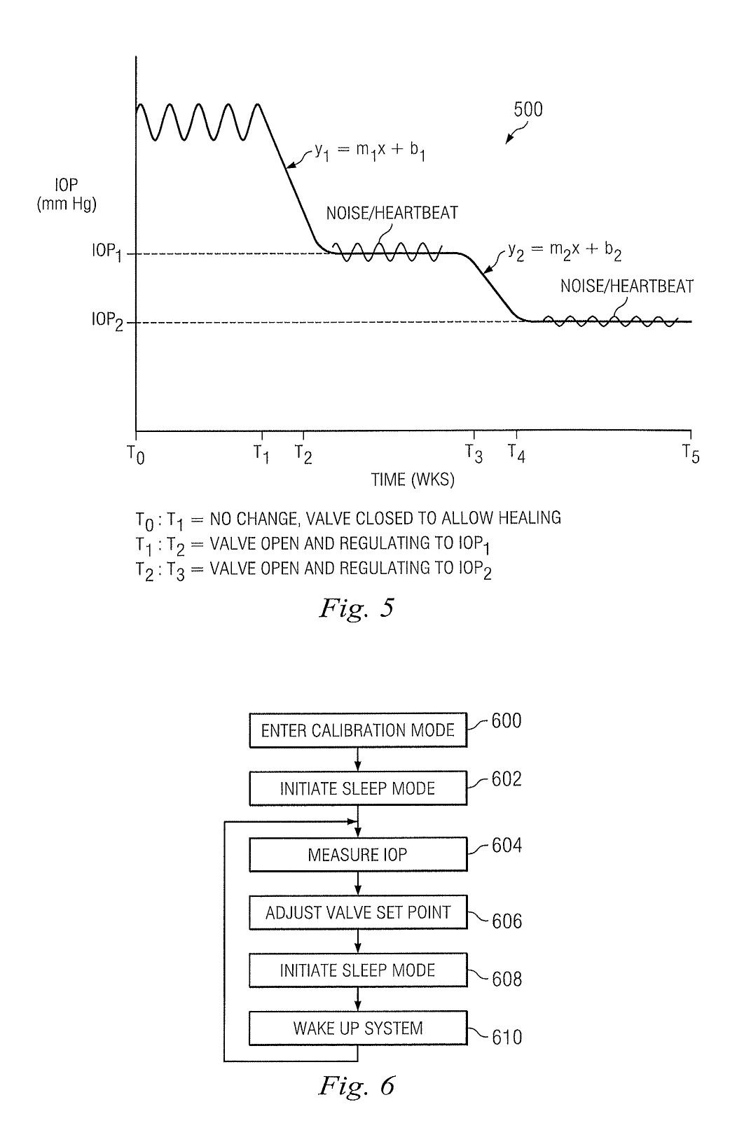

FIG. 5 shows a graph 500 indicative of an exemplary treatment algorithm that may be stored for selection and for treatment of an ocular condition. In this example, the graph 500 shows the treatment plan relative to IOP over a time period starting at the time the implant 300 is implanted in the patient's eye. In FIG. 5, immediately following implantation, from the time T₀ to T₁, the flow system 212 prevents drainage from the anterior chamber of the eye. This may be accomplished by simply maintaining a valve forming a part of the flow system 212 in a closed state or condition. This may provide the eye an opportunity to recover from the implant procedure prior to initiating drainage to alleviate elevated IOP conditions. This may result in better healing and fewer complications than may otherwise occur as a result of the initial surgical procedure.

During the period of time T₁ to T₂, the processor 206 controls the flow system 212 to gradually decrease the IOP to target value IOP₁. The rate of change from the IOP value in the prior period T₀ to T₁ to the target IOP₁ may be set using the slope-intercept equation y=mx+b, and may vary depending on the equation and the desired change. It should be noted that y=mx+b is a known linear equation and is used here as an example that may be substituted with more complex nonlinear or discontinuous equations to accommodate different needs.

During the period of time T₂ to T₃, the processor 206 controls the flow system 212 maintain the actual IOP in line with the target value IOP₁. It may be do this based on readings from the sensor system 204 including readings received from the sensors P1, P2, and P3. By calculating the IOP based on the sensor readings, and comparing the calculated IOP to the target value IOP_{I}, the IOP control system 200 can determine whether to further control the flow system 212 to increase or decrease flow to maintain the desired IOP. For example, the valves in the flow system 212 may be opened wider or closed more, and a pump may or may not be activated to control the flow and regulate the IOP, depending on the data measured by the sensors.

During the period of time T₃ to T₄, the flow system 212 is controlled to gradually decrease the IOP to target value IOP₂. The rate of change from the IOP value in the prior period T₃ to T₄ to the target IOP₂ may be set using the slope-intercept equation y=mx+b, and may vary depending on the equation and the desired change.

Finally, during the period of time T₄ to T₅, which may last until the time the implant is surgically removed, the processor 206 controls the flow system 212 to maintain the actual IOP in line with the target value IOP₂.

A corresponding flow chart representing control logic of a treatment algorithm is shown in FIG. 6 to further explain the exemplary treatment algorithm. The flow chart 600 represents control logic that may be used to implement one of the treatment algorithms, such as the treatment algorithm represented in FIG. 5.

The method in FIG. 6 may be a continuation of the method performed and described with reference to FIG. 4. In FIG. 6, at a step 600, the system 200 enters a calibration mode. In the calibration mode, the system 200 retrieves from memory the selected treatment algorithm and loads the selected treatment algorithm for execution by the processor 206. It may then begin to operate the control system in accordance with the selected treatment algorithm. Steps 602-610 describe the execution or the process carried out as a result of a particular treatment algorithm, such as the algorithm described above with reference to FIG. 5.

In accordance with the treatment algorithm in FIG. 5, the control system 200 operates first in a mode that prevents drainage flow through the implant 300 from the anterior chamber. Accordingly, the system 200 may close the valve in the flow system 212 or if closed is the default position, may take no action on the flow system 212. Consistent with this, in step 602, the system 200 may enter into a sleep mode since there is no contemplated action during the period T0-T1 in FIG. 5. The sleep mode may minimize power consumption and provide a maximum life span for the implantable device 300. In the sleep mode, an internal timer may track the time period until time T2. At time T2, the implant may awake and measure IOP as indicated at step 604. It should be noted that the sleep mode may be used at various time throughout the life of the implant depending on the treatment algorithm chosen or programmed.

Based on this measurement, the processor 206 may adjust a valve set point of the flow system 212 in order to achieve the desired IOP at a step 606. In some treatment algorithms, such as the one disclosed with reference to FIG. 5, the valve set point may be adjusted gradually over a period of time to modify the IOP to a desired value, such as IOP₁ shown in FIG. 5.

At step 608, after the valve set point is modified to maintain the IOP at a desired value, the system 200 initiates a sleep mode for a period of time corresponding to T2-T3 in FIG. 5. The sleep mode may be a short period of time, such a period of 2-3 hours or a long period of time, such as a period of a week or more, although longer and shorter times are contemplated and will vary depending on the treatment algorithm. During this time, the implant 300 is maintained in a stable condition, without response to any pressure readings. At a step 610, after the period of time designated by the treatment algorithm, the system wakes up. Once it is awake, they treatment algorithm returns to step 604 to measure IOP. It then continues to carry out the method in a loop to maintain the IOP at a desired target or target range according to the selected treatment algorithm or profile.

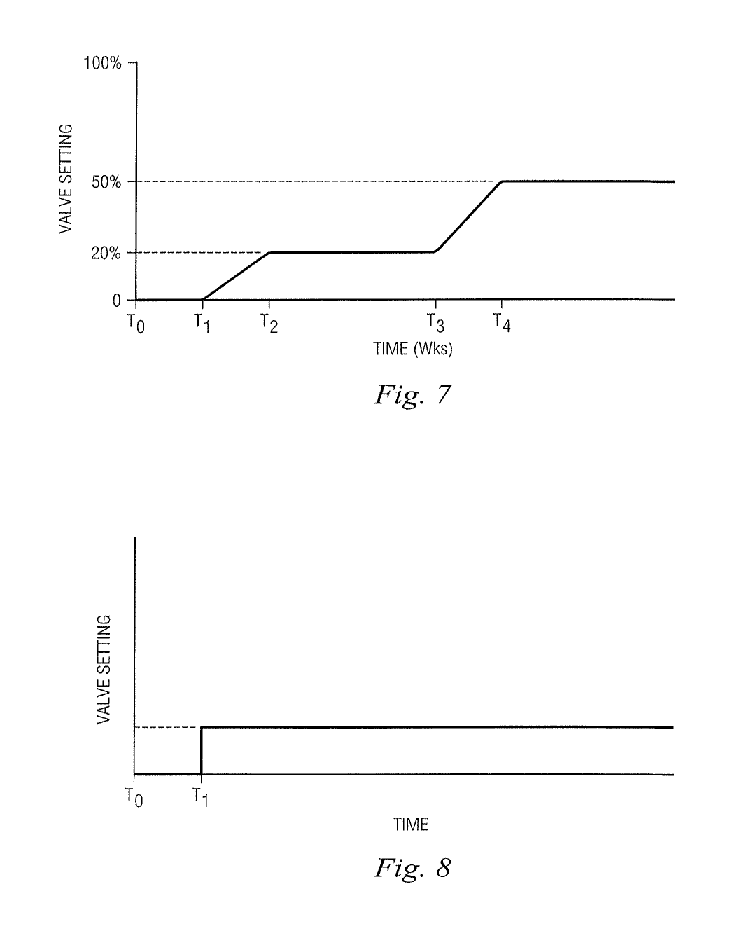

In an alternative embodiment, the treatment algorithm controls flow based on settings of the flow system 212 in an open loop configuration where the system relies upon settings of elements of the flow system 212 instead of, in place of, or in addition to, settings based on detected pressure readings as indicative of IOP. In this embodiment, the control system 200 may control flow based on settings of the components, such as a valve setting. One example of such a treatment algorithm is described with reference to FIG. 7. FIG. 7 shows a graph embodying another exemplary treatment algorithm showing the valve setting (instead of the IOP target) as the driving factor controlling the system 200. Here the valve setting is shown as a percentage of amount that the valve is open, over time.

In FIG. 7, immediately following implantation, from the Time T₀ to T₁, the flow system 212 prevents drainage from the anterior chamber similar to the algorithm discussed above with reference to FIG. 5. During the period of time T₁ to T₂, the processor 206 controls the flow system 212 to gradually open a valve in the flow system 212 to increase drainage flow until the valve is open a target percentage amount. This target amount may be any amount based on the treatment algorithm. In the treatment algorithm shown in FIG. 7, the target amount is 20% open. In another treatment algorithm or profile, the target amount is 30% open. Naturally, other amounts are contemplated. This gradual increase may occur continuously over the period of time T₁ to T₂ in one embodiment. In another embodiment, in order to conserve power, the increase may be accomplished in steps, where power is required only for the step, and then the setting may be maintained for a period of time.

During the period of time T₂ to T₃, the processor makes no additional adjustments to the flow system 212, thereby maintaining the settings for the flow system.

During the period of time T₃ to T₄, the processor adjusts the flow system 212 further, increasing the drainage capacity of the implant by opening the valve further to permit additional drainage. In this example, the setting during the period of time T₃ to T₄ is 50% open. Other amounts are contemplated depending on the stored treatment algorithm. During the time period after T₄, the valve is maintained at its set position in accordance with the treatment algorithm.

FIG. 8 shows a graph representing another treatment algorithm. The treatment algorithm in FIG. 8 includes an abrupt adjustment or change (i.e. discontinuous function) in the amount that the valve is opened from a closed position at period of time T₁. After the adjustment at time T₁, the setting is maintained relatively constant.

As described above, the implant 300 may store one or more of the treatment algorithms to provide a desired treatment profile for a particular patient. A surgeon may select the desired treatment algorithm based on any number of factors.

In one embodiment, the surgeon may modify the treatment algorithm by increasing the levels of IOP₁, IOP₂, percentage open, or other amounts. For example, one embodiment of the system allows a surgeon to increase or decrease the time period of T₀ to T₁ from a default value. Another embodiment permits a surgeon to incrementally shift the treatment algorithm upward or downward along the y-axis in the graphs of FIGS. 5, 7, and 8. Theoretically, the surgeon would be able to input any treatment algorithm that falls within the capabilities of the system.

In one example, the treatment algorithm is arranged to operate at a first IOP setting during a morning and to operate at a second IOP setting later in the day. The system may reset at a particular time and run the same program the next day. Accordingly, the treatment algorithm may make daily adjustments based on the time of day, readings from the sensor system 204, or other factors.

As described above, the implant may include any number of treatment algorithms to treat a medical condition. For example, the flow system 212 can be adjusted to maintain a particular IOP (like an IOP of 15 mm Hg). Flow system 212 may be opened at desirable times, such as, for example, more at night than during the day to maintain a particular IOP. In other embodiments, an IOP drop can be controlled by the flow system 212. The flow system 212 can be adjusted to permit a gradual drop in IOP based on readings from pressure sensors P1 and P3. In some embodiments, the physician would be able to set the high/low IOP thresholds wirelessly to meet each patient's specific requirements.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An apparatus for treatment of a medical condition of a patient to provide drainage from a region of a patient to a drainage location, comprising:
a memory having a plurality of different selectable treatment algorithms stored therein for treating a medical condition;
a processor associated with the memory and configured to execute one of the different treatment algorithms; and
a flow system controllable by the processor to regulate drainage of fluid from a body portion having a medical condition, the flow system being controllable according to a selected algorithm of said different selectable treatment algorithms.

2. The apparatus of claim 1, further comprising an implantable medical device for treating an ocular condition, wherein the processor is carried on the implantable medical device.

3. The apparatus of claim 1, wherein each of the plurality of treatment algorithms comprises a plurality of periods of time and target settings corresponding to the periods of time.

4. The apparatus of claim 1, wherein the flow system comprises one of a valve and a pump, and the processor controls a setting on said valve or pump.

5. The apparatus of claim 1, further comprising a sensor system detecting pressures within the body portion having a medical condition.

6. The apparatus of claim 5, wherein information from the sensor system enables calculation of an IOP of an eye.

7. The apparatus of claim 1, wherein the plurality of different selectable treatment algorithms comprise target IOP values, the processor being configured to control the flow system to obtain the target IOP value for the body portion.

8. The apparatus of claim 1, wherein the plurality of different selectable treatment algorithms comprise target valve settings, the processor being configured to control the flow system to obtain the target valve setting.

9. The apparatus of claim 1, comprising a drainage tube connected to the flow system and sized to extend into the anterior chamber of the eye.

10. The apparatus of claim 1, wherein at least one of the control algorithms prevents drainage for a period of time after implantation.

11. A control system for treatment of an ocular condition of a patient to provide drainage from an anterior chamber of the eye to a drainage location, comprising:
a memory having a plurality of different selectable treatment algorithms stored therein for treating an ocular condition;
a processor associated with the memory and configured to execute one of the different treatment algorithms; and
a sensor system configured to detect a pressure representative of an anterior chamber and configured to detect a pressure representative of atmospheric pressure, the processor being configured to generate control signals based on a selected one of the different treatment algorithms and based on the detected pressures.

12. The control system of claim 11, comprising a flow system comprising one of a valve and a pump controllable by the processor to regulate drainage of fluid from an eye, the flow system being controllable according to said selected one of said different selectable treatment algorithms.

13. The control system of claim 12, wherein the plurality of different selectable treatment algorithms comprises at least treatment algorithm that includes instructions for adjusting the flow system based on target valve settings.

14. The control system of claim 11, comprising a data transmission module configured to receive one of:
a) a selection of said selected one of the different treatment algorithms; and
b) the selected one of the different treatment algorithms.

15. The control system of claim 14, wherein the data transmission module is structurally configured to receive said selection or said selected algorithm via remote transmission for programming the system.

16. The control system of claim 11, further comprising an input interface for receiving an input selecting the selected algorithm of said different selectable treatment algorithms.

17. A method comprising:
storing a plurality of selectable treatment algorithms in a memory;
receiving an input selecting one of the selectable treatment algorithms of the plurality of selectable treatment algorithms in a memory; and
controlling a flow system to regulate drainage of fluid from a body portion having a medical condition based on the selected one of the selectable treatment algorithms.

18. The method of claim 17, comprising incrementally adjusting the treatment algorithm.

19. The method of claim 17, wherein the treatment algorithm includes a target IOP level.

20. The method of claim 17, wherein the treatment algorithm comprises a target open amount of an adjustable valve.

21. The method of claim 17, comprising communicating the selected one of the selectable treatment algorithms to the processor, the processor performing the controlling step.

22. The method of claim 17, further comprising receiving an input from a health care provider to modify the selected treatment algorithm, and wherein controlling a flow system based on the selected one of the selectable treatment algorithms includes controlling the flow system based on the modified algorithm.
